(19) United States
(12) Patent Application Publication
(10) Pub. No.: US 2007/0027381 A1
(43) Pub. Date: Feb. 1, 2007

(54) Inserter and methods of use

(75) Inventor: Gary Ashley Stafford, Hayward, CA (US)

Correspondence Address: JACKSON & CO., LLP, 6114 LA SALLE AVENUE, SUITE 507, OAKLAND, CA, 94611-2802, US

(73) Assignee: TheraSense, Inc., Alameda, CA (US)

(21) Appl. No.: 11/192,773

(22) Filed: Jul. 29, 2005

(51) Int. Cl. A61B 5/05

(52) U.S. Cl. 600/347; 128/903; 600/345

(57) ABSTRACT

Method and apparatus for providing a housing integrated with a sensor introducer and a sensor in analyte monitoring system to deploy the sensor and retain the introducer within the housing upon sensor deployment, and mounting a transmitter to the housing to receive the sensor data is described. The housing may be placed on the skin of a patient and a spring biased mechanism, separately provided and coupled to the introducer or by integrally configuring the introducer, triggers the introducer to deploy the insertion mechanism for placement of the sensor at a predetermined depth under the skin of the patient.

### BACKGROUND

The present invention relates to data monitoring and detection systems. More specifically, the present invention relates to method and apparatus for providing a transcutaneous sensor insertion into a patient for use in analyte monitoring systems including continuous glucose monitoring systems.

Continuous glucose monitoring systems generally include a sensor such as a subcutaneous analyte sensor for detecting analyte levels such as blood glucose levels, a transmitter (such as an RF transmitter) in communication with the sensor and configured to receive the sensor signals and to transmit them to a corresponding receiver unit by for example, using RF data transmission protocol. The receiver may be operatively coupled to a blood glucose monitor that performs blood glucose related calculations and data analysis.

The transmitter may be mounted or adhered to the skin of a patient and also in signal communication with the sensor, a portion of which may be implanted into the skin of the patient. Generally, the sensor is configured to detect and measure the blood glucose levels of the patient over a predetermined period of time, and the transmitter is configured to transmit the measured blood glucose levels over the predetermined period of time for further analysis. To initially set up the sensor so that the sensor contacts and electrodes are in fluid contact with the patient's analyte fluids, it is important to properly insert the sensor through the patient's skin and securely retain the sensor during the time that the sensor is configured to detect analyte levels. In addition to accurate positioning of the sensor through the skin of the patient, it is important to minimize the level of pain associated with the insertion of the sensor through the patient's skin.

In view of the foregoing, it would be desirable to have method and apparatus which would allow for accurate and easy insertion of the sensor through the skin of a patient or otherwise to properly position the sensor transcutaneously so that the sensor maybe configured to detect analyte levels of the patient. Also, it would be desirable to have a method and apparatus to have an integrated sensor insertion mechanism and transmitter mount or housing portion which may be mounted on the patient's skin with ease and relative little pain to the patient.

### SUMMARY OF THE INVENTION

In one embodiment, there is provided a rotary inserter configuration incorporating an introducer for deployment of a sensor such as an electrochemical sensor through the skin of a patient to transcutaneously place the sensor in fluid contact with the patient's analyte. The rotary inserter configuration in one embodiment includes a spring biased insertion mechanism which, upon actuation or trigger, is configured to deploy the introducer and the sensor, and upon deployment of the sensor in the patient, to retract the introducer from the patient and within the housing so that it does not interfere with the analyte monitoring. In one embodiment, the insertion mechanism may also include a “Scotch-Yoke” type mechanism configured to translate rotational motion into linear motion. Alternatively, the insertion mechanism may include gears and/or a cam as well.

In a further embodiment of the present invention, the rotary inserter configuration is integrated with a mounting unit of a sensor control unit, or a base housing which is configured to receive a data transmitter (or a transceiver). As such, a single device which incorporates the sensor insertion mechanism as well as providing the support structure for mounting the transmitter to a patient is provided. The data transmitter in one embodiment is configured for electrical communication with the sensor, where the sensor is configured to detect the patient's analyte level, and the transmitter configured to transmit (wirelessly or otherwise) to a monitoring unit such as a glucose monitor unit or an insulin pump.

In this manner, in accordance with the various embodiments of the present invention, the sensor may be deployed using a trigger mechanism of a rotary inserter configuration that may be actuated by a simple rotary type movement of the insertion mechanism, and which is configured to retain the introducer within the housing so as to be discarded with the housing and/or with the replacement of the sensor.

### BRIEF DESCRIPTION OF THE DRAWINGS

FIG. 1 illustrates a perspective view of the rotary inserter configuration in pre-deployed position in accordance with one embodiment of the present invention;

FIGS. 2A-2B illustrate a top planar view of the rotary inserter configuration shown in FIG. 1;

FIG. 3 illustrates the introducer coupled to an insertion mechanism in the pre-deployed position as shown in FIG. 1 in accordance with one embodiment of the present invention;

FIG. 4 illustrates a perspective view of the rotary inserter configuration in a deployment position in accordance with one embodiment of the present invention;

FIG. 5 illustrates the introducer coupled to the insertion mechanism in the deployment position shown in FIG. 4 in accordance with one embodiment of the present invention;

FIG. 6 illustrates a perspective view of the rotary inserter configuration in deployed position in accordance with one embodiment of the present invention;

FIG. 7 illustrates a side planar view of the rotary inserter configuration in the deployed position shown in FIG. 6 in accordance with one embodiment of the present invention;

FIG. 8 illustrates a front planar view of a coil spring insertion mechanism in accordance with a further embodiment of the present invention;

FIG. 9 illustrates a perspective view of the coil spring insertion mechanism of FIG. 8 in accordance with one embodiment of the present invention;

FIG. 10 illustrates a side planar view of the coil spring insertion mechanism of FIG. 8 in accordance with one embodiment of the present invention;

FIG. 11 illustrates a front planar view of a flexible introducer insertion mechanism in accordance with still a further embodiment of the present invention;

FIG. 12 illustrates a perspective view of the flexible introducer insertion mechanism in accordance with one embodiment of the present invention; and

FIG. 13 illustrates a side planar view of the flexible introducer insertion mechanism in accordance with one embodiment of the present invention.

### DETAILED DESCRIPTION

FIG. 1 illustrates a perspective view of the rotary inserter configuration in pre-deployed position in accordance with one embodiment of the present invention. Referring to the Figure, rotary inserter configuration 100 in one embodiment of the present invention includes a housing (or mount) 101 configured substantially as shown in the Figure, and which includes a base portion at a substantially right angle to a sensor insertion portion. As shown in the Figure, the sensor insertion portion of the housing 101 includes a cavity 105 that is configured to substantially house an analyte sensor 104, an introducer 103 and an insertion mechanism 102 as discussed in further detail below. Referring again to FIG. 1, it can be seen that in a pre-deployed position, the sensor 104, the introducer 103 and the insertion mechanism 102 are substantially completely retained within the cavity 105 of the insertion portion of the housing. However, in certain embodiments some or all of these may be only partially retained within cavity 105.

Moreover, it can be further seen from the Figure that the introducer 103 is physically coupled to the senor 104 such that, when the insertion mechanism 102 is deployed, the introducer 103 is configured to be physically displaced with the sensor 104 so as to deploy and position the sensor 104 in a desired location (for example, at least a portion of the sensor in contact with the subcutaneous tissue under the skin of a patient).

Additionally, the insertion mechanism 102 as shown in FIG. 1 is configured to be physically or mechanically coupled to the introducer 103 via a coupling mechanism 106. More specifically, as discussed in further detail below, in one embodiment of the present invention, the insertion mechanism 102 may be substantially configured as, for example, a circular thumb wheel mechanism or the like rotatably mounted at its center position (or otherwise) within the insertion portion of the housing 101. In such embodiments, the insertion mechanism 102 may be easily and readily rotated by the movement of a user's thumb or any other finger along the opening portion of the cavity 105 as shown. In other words, in certain embodiments, the circumferential edge portion of the insertion mechanism 102 may be positioned within the cavity so as to be physically accessible by a patient.

Moreover, in certain embodiments, the insertion mechanism 102 in one embodiment is provided with a coupling mechanism 106 which is configured to physically couple to the introducer 103 as shown in FIG. 1. In this manner, as will be discussed in further detail below, the rotatable movement of the insertion mechanism 102 is configured to correspondingly displace the position of the introducer 103 within the cavity 105 of the insertion portion of the housing 101. In turn, the displacement of the introducer 103 will correspondingly move the sensor 104 within the cavity 105 so as to position the sensor 104 in the desired location.

Referring yet again to the Figure, while a pin type coupling mechanism 106 is shown, within the scope of the present invention, the introducer 103 may be coupled in many different ways to couple to the insertion mechanism 102 so that the position and thus the deployment of the introducer 103 may be controlled by the insertion mechanism 102. For example, within the scope of the present invention, the coupling mechanism 106 may include, but is not limited to, a hinged or pivotable coupling mechanism, or the like. In addition, within the scope of the present invention, the insertion mechanism 102 may be spring biased (or spring loaded) so that in the pre- deployed position as shown in FIG. 1, the insertion mechanism 102 may include a trigger function which includes the bias of the spring for the insertion mechanism to be in a coiled position. Thereafter, upon user or patient activation of the trigger function by, for example, the patient's movement of the patient's finger or thumb over the cavity 105 of the housing 101 so as to displace the insertion mechanism 102, the spring loaded insertion mechanism 102 may be triggered so as to rotatably displace the introducer 103 including the sensor 104 to the deployment position in the cavity 105.

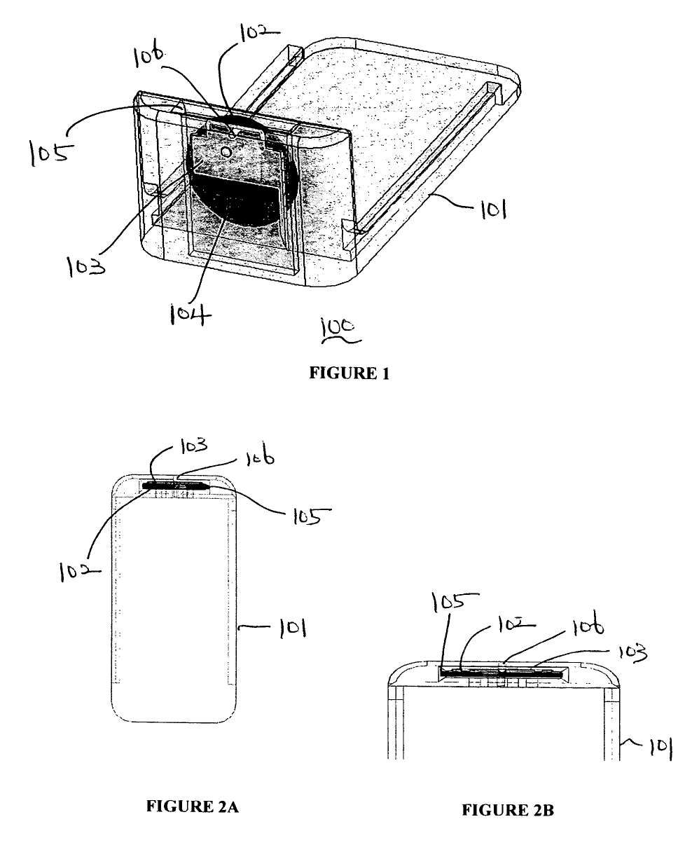

FIGS. 2A-2B illustrate a top planar view of the rotary inserter configuration shown in FIG. 1. Referring to the Figures, in can be seen that the cavity 105 may be configured in one embodiment to substantially physically retain the introducer 103, the sensor 104 and the insertion mechanism 102. Moreover, while not shown in the Figures, a transmitter unit in one embodiment is configured to be removably placed on the base portion of the housing 101 so that when the transmitter unit is positioned substantially on the base portion of the housing, the transmitter unit is configured to be in electrical contact with the sensor 104. In this manner, the detected analyte levels from the sensor 104 may be transmitted to the transmitter unit, which is, in one embodiment, configured to wirelessly transmit the sensor signals to a receiver unit such as a glucose monitor unit, an insulin pump unit, or a computer terminal.

In certain embodiments, the transmitter may be integrated into the base portion of the housing, e.g., integrated in or on the base portion, so as to provide a unitary piece of construction. Such embodiments reduce the number of separate components and reduce the number of steps a user performs by omitting or minimizing the steps of attaching or mounting a transmitter to the base portion. More specifically, in one embodiment, the transmitter may be partially attached before the insertion of the sensor 104. Indeed, these steps can be performed with less difficulty and with more reliability, as both hands of the patient can be used for tabletop assembly before the housing 101 is applied to the skin. After the insertion of the sensor 104, the transmitter may be easily moved to the final position using one hand. Indeed, the steps of attaching and detaching an inserter in one embodiment of the present invention is eliminated because the insertion mechanism in accordance with the various embodiments of the present invention is integrated and built into the housing 101 of the transmitter. Additional information on the detection, monitoring and analysis of analyte levels are described in further detail in U.S. Pat. No. 6,175,752 entitled “Analyte Monitoring Device and Methods of Use” the disclosure of which is incorporated herein by reference for all purposes.

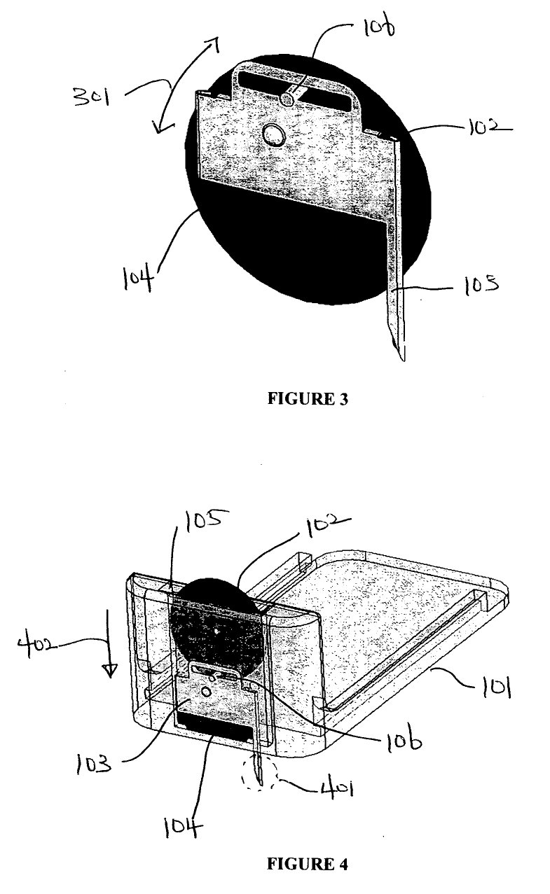

FIG. 3 illustrates the introducer coupled to an insertion mechanism in the pre- deployed position as shown in FIG. 1 in accordance with one embodiment of the present invention. Referring to the Figure, the insertion mechanism 102 and the introducer 103 coupled with the sensor 104 is shown outside of the cavity 105 of the insertion portion of the housing 101. It can be seen that as discussed in detail above, the coupling mechanism 106 of the insertion mechanism 102 is configured in one embodiment to mechanically couple with the introducer 103.

In this manner, when the patient rotates the insertion mechanism 102 along the rotational direction of the insertion mechanism 102 as shown by the directional arrow 301, the introducer 103 and the sensor 104 are configured to be displaced to the desired location. More specifically, as discussed in further detail below, the rotational movement of the insertion mechanism 102 in one embodiment is configured to displace the introducer 103 so that the introducer 103 deploys the sensor 104 through the skin of the patient (or the surface on which the housing 101) is placed, e.g., to transcutaneously position the sensor with respect to the skin of the patient.

FIG. 4 illustrates a perspective view of the rotary inserter configuration in a deployment position in accordance with one embodiment of the present invention. Referring to the Figure, it can be seen that in the deployment position, the introducer tip portion 401 is configured to protrude beyond the bottom surface of the housing 101 so as to extend out of the cavity 105 and thus pierce the skin of the patient to which the bottom portion of the housing 101 is in contact. More specifically, in the deployment position as shown in FIG. 4, the insertion mechanism 102 when actuated, in one embodiment of the present invention, is configured to,displace the introducer 103 and the sensor 104 in a downward direction within the cavity 105, and as shown by the directional arrow 402 shown in FIG. 4.

Indeed, as can be seen from FIG. 4, the deployment position of the rotary inserter configuration in one embodiment of the present invention is configured to physically translate the position of the introducer 103 and the sensor 104 such that the introducer 103 is placed in motion to deploy the sensor 104 to the desired location relative to the housing 101, and also, relative to the patient's skin or body.

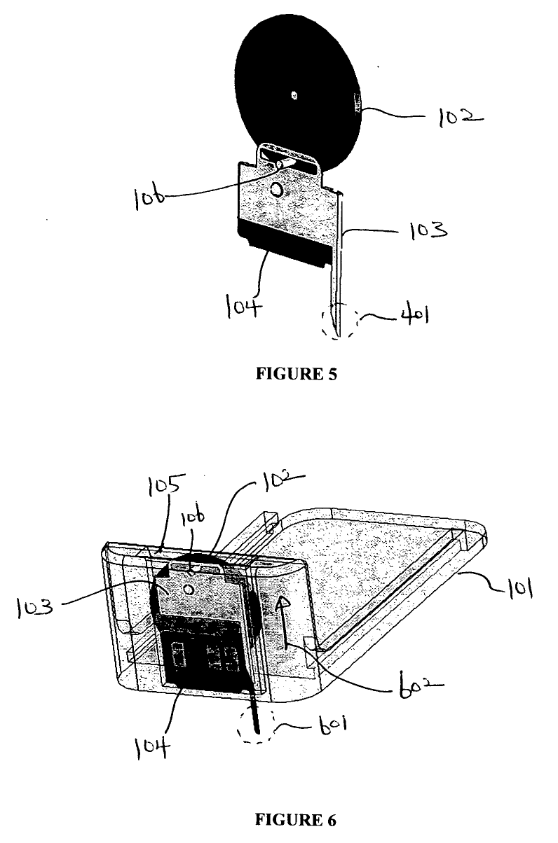

FIG. 5 illustrates the introducer coupled to the insertion mechanism in the deployment position shown in FIG. 4 in accordance with one embodiment of the present invention. Referring to the Figure, similar to the embodiment shown in FIG. 3, the insertion mechanism 102 coupled to the introducer 103 and the sensor 104 is shown outside of the cavity 105 of the housing 101. As shown in the Figure, it can be seen that the coupling mechanism 106 physically coupling the introducer 103 to the insertion mechanism 102, is displaced so as to fully deploy the sensor 104 coupled to the introducer 103.

In other words, at the deployment position shown in FIG. 5, the tip portion 401 of the introducer 103 which in one embodiment is configured to guide a portion of the sensor 104, is configured to correspondingly deploy the sensor 104 by guiding the portion of the sensor 104 with the force provided by the insertion mechanism 102 in displacing the introducer 103. In one embodiment, as discussed above, the insertion mechanism 102 may be spring loaded so as to provide the force needed to trigger the introducer 103 to be deployed through a surface such as a patient's skin.

FIG. 6 illustrates a perspective view of the rotary inserter configuration in deployed position in accordance with one embodiment of the present invention. Referring to the Figure, it can be seen that upon the deployment of the introducer 103 and correspondingly the sensor 104, the insertion mechanism 102 is configured to be displaced within the cavity 105 of the housing to return to the initial pre-deployment position. This can also be seen by the directional arrow 602 shown in FIG. 6 which illustrates the direction along which the introducer 103 is configured to move.

Referring back to FIG. 6, in the deployed position of the rotary inserter configuration, the sensor 104 is configured to be substantially and permanently displaced such that the sensor portion which is configured to be placed in fluid contact with the patient's analyte-containing fluid is thus position as such and out of the cavity 105 of the housing 101. This is shown in FIG. 6 where tip portion 601 of the sensor 104 in the deployed position is securely positioned out of the cavity 105 of the housing 101.

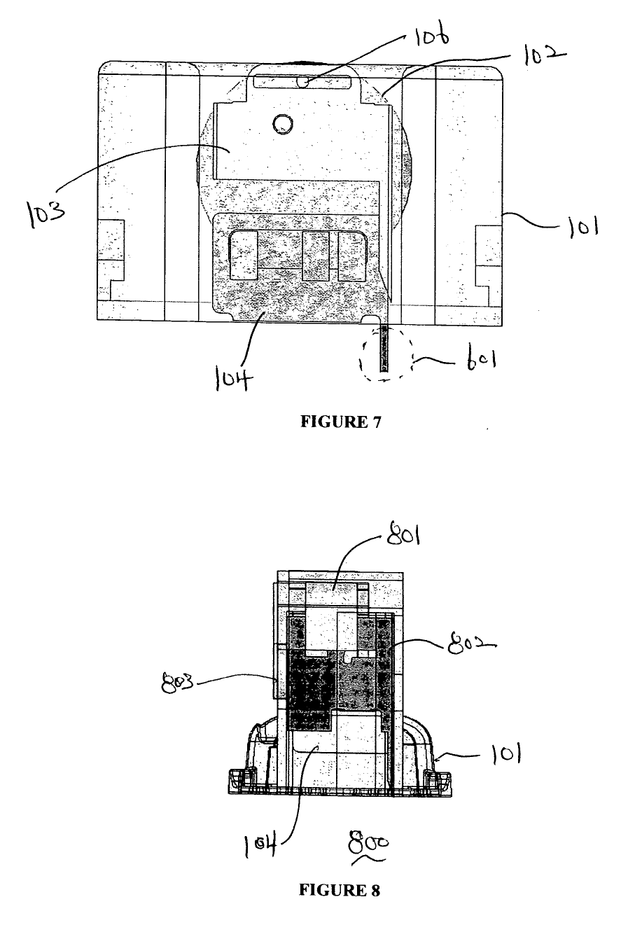

The relative position of the insertion mechanism 102, the introducer 103 and the sensor 104 upon deployment and in deployed position can be also seen in FIG. 7 which illustrates a side planar view of the rotary inserter configuration in the deployed position shown in FIG. 6 in accordance with one embodiment of the present invention. In this manner, as shown in the Figures and in accordance with one embodiment of the present invention, the insertion mechanism 102 is configured to deploy the introducer 103 and the sensor 104 by a simple trigger function of the patient's rotatable displacement of the insertion mechanism 102.

Upon actuation of the trigger function, the spring biased insertion mechanism 102 in one embodiment is configured to rapidly displace the introducer 103 (and thus the sensor) through the skin of a patient, so as to pierce the skin surface, and deploy the sensor 104, and also to retract the introducer 103 out of the patient, leaving behind the sensor 104 in, for example, fluid contact with the patient's analyte levels. Thereafter, the sensor 104 positioned within the housing 101 is configured to transmit analyte levels to the transmitter unit mounted onto the housing. It should be noted that in one embodiment, the speed of insertion of the introducer 103 and the sensor 104 is substantially a function of the speed at which the patient manipulates or activates the insertion mechanism 102.

Moreover, while a spring loaded mechanism is discussed, within the scope of the present invention, other equivalent trigger mechanism may be used to deploy the introducer 103, and thus to provide the functionality of the insertion mechanism.

FIG. 8 illustrates a front planar view of a coil spring insertion mechanism in accordance with a further embodiment of the present invention. Referring to the Figure, the coil spring insertion mechanism 800 in one embodiment of the present invention includes a torsion spring 801 which is coupled to an introducer 802, and which in one embodiment is configured to be controlled by a lever 803 mounted thereon. Referring to FIG. 8, it can be seen that the torsion spring 801 and the introducer 802 as well as the lever 803 is substantially positioned within the insertion portion of the housing 101. Furthermore, it can be seen that the analyte sensor 104 is integrally provided within the sensor insertion portion of the housing 101, and further, is coupled to the introducer 802 such that, when the lever 803 is actuated (for example, by the user or patient), the sensor 104 is configured to be displaced out of the housing 101 by the movement of the introducer 802 under the force of the torsion spring 801.

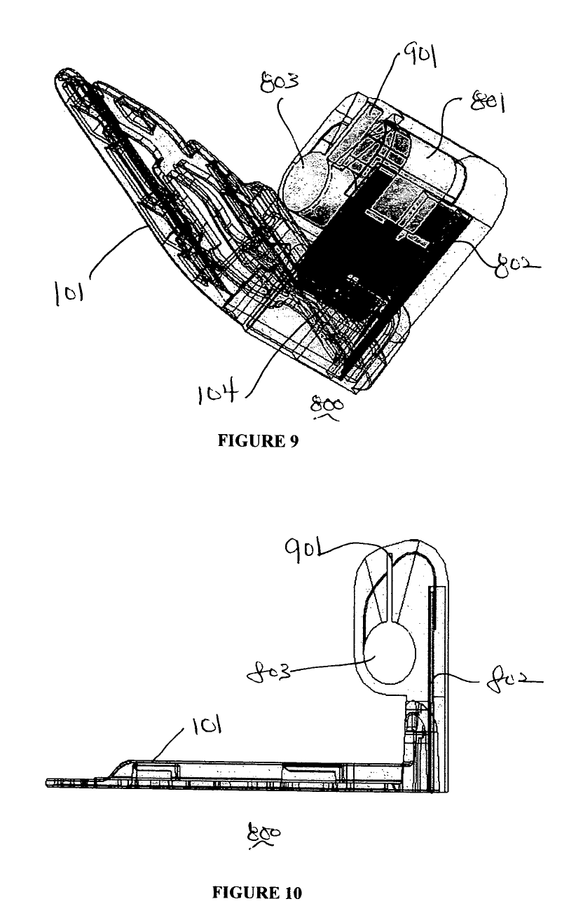

FIGS. 9 and 10 illustrate a perspective view and a side planar view, respectively, of the coil spring insertion mechanism of FIG. 8 in accordance with one embodiment of the present invention. Referring to the Figures, as can be seen, a handle portion 901 may be integrally provided to the lever 803 and positioned substantially partially outside of the housing 101 to provide access to the patient in order to actuate the lever 803 to trigger the introducer 802 so as to place the sensor 104 transcutaneously to the skin of the patient.

Referring back to FIGS. 8-11, the coil spring insertion mechanism 800 in one embodiment of the present invention may be provided to the patient in fully assembled configuration with the sensor 104 and skin attachment or adhesive (not shown). In this manner, the patient may easily and readily place the coil spring insertion mechanism 800 onto the skin of the patient at the desired site, and upon activating the lever 803 by, for example, pulling down on the lever 901), the sensor 104 is introduced through the skin of the patient at the desired site guided by the introducer 802.

In the manner described above, in accordance with one embodiment of the present invention, the coil spring insertion mechanism 800 including introducer 802 coupled to the torsion spring 801 allows the sensor 104 to be substantially precisely guided through the opening (not shown) of the housing 101 and transcutaneously implanted into the patient to a desired specified depth. More specifically, when the lever 803 is pushed back, the torsion spring 801 triggers the introducer 802 to be driven downward into the skin of the patient. When the lever 803 is released thereafter, the torsion spring 801 allows the introducer 802 to retract out of the skin and remain in the up position, having introduced the sensor 104 through the skin of the patient. Thereafter, the transmitter (not shown) may be mounted and positioned onto the housing 101 so that the sensor 104 may establish an electrical communication with the transmitter.

In the manner described above, in accordance with one embodiment of the present invention, such “on-board” insertion configuration of analyte sensors eliminates the need for a sensor delivery unit (such as a separate insertion device), and thereby reducing the associated material costs, weight, packaging, handling, as well as disposal thereof. Additionally, the number of steps that are necessary for the patient to perform to introduce and position the analyte sensor is reduced which provides significant advantages, especially in continuous monitoring systems where the sensor typically requires replacement at a predetermined interval.

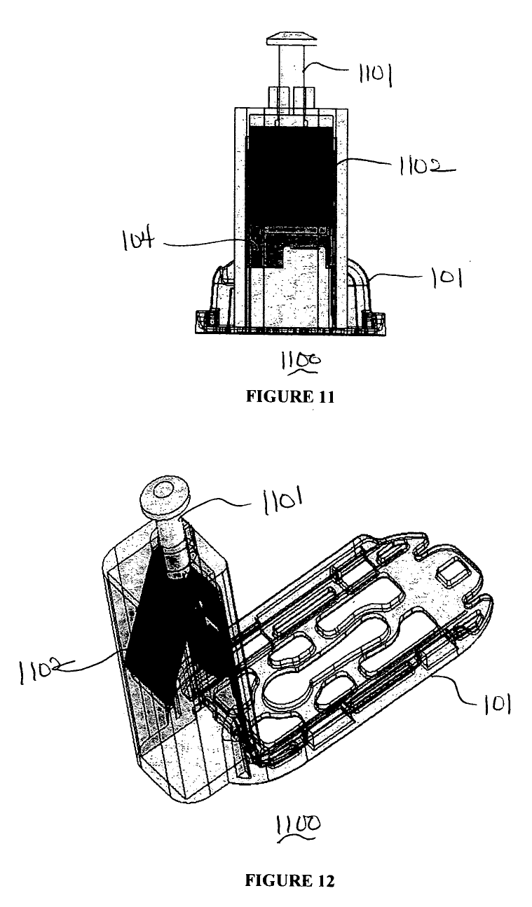

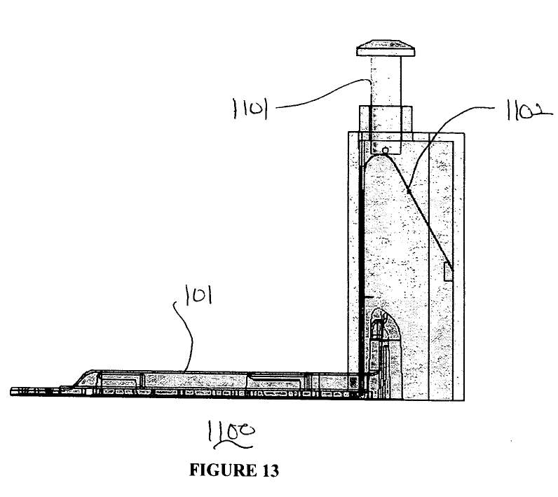

FIG. 11 illustrates a front planar view of a flexible introducer insertion mechanism in accordance with still a further embodiment of the present invention. Additionally, FIGS. 12 and 13 respectively illustrate a perspective view and a side planar view of the flexible introducer insertion mechanism in accordance with one embodiment of the present invention. Referring to the Figures, the flexible introducer insertion mechanism 1100 includes a plunger 1101 coupled to an introducer 1102 (which is coupled to the sensor 104 for placement) provided within the housing 101.

In particular, as can be seen from FIGS. 11-13, when the plunger 1101 is pushed down by the patient, the introducer 1102 is configured to drive the introducer 1102 and the sensor 104 into the skin of the patient to be placed at a predetermined depth under the skin of the patient. Thereafter, the release of the plunger 1101 allows the introducer 1102 to be retracted from the deployed position and return to the original pre-deployment position within the housing 101. In other words, in the embodiment shown in FIGS. 11-13, the actuation of the plunger 1101 is configured to drive the introducer 1102 and the sensor 104 through the skin of the patient so as to place the sensor 104 transcutaneously, for example, through the patient's skin at a predetermined and precise depth.

Referring back to the Figures, once the introducer 1102 is in the up position within the housing and withdrawn out of the patient (leaving behind the sensor 104), the plunger in one embodiment may be configured to be permanently removed from the housing 101. For example, in one embodiment, the plunger 1101 may be configured to be snapped off, twisted or broken off from the housing 101 so that it is less cumbersome for the patient to have the housing 101 mounted onto the skin for a predetermined period of time such as 3 to 5 days during which the sensor 104 is configured to continuously, semi-continuously, intermittently or intermittently and repeatedly detect the patient's analyte level (for example, glucose level).

In the manner described above, in accordance with the various embodiments of the present invention, an integrated introducer and transmitter mount may be provided. More specifically, it is possible for the diabetic patients to have to use one less device in order to monitor the glucose levels for insulin therapy and management. Additionally, since the introducer 103 in one embodiment is substantially and completely housed within the cavity 105 of the housing 101 upon full deployment of the sensor 104, the patient likewise need not worry about disposing of the introducer 103 including its sharp and potentially dangerous edges and/or segments. Moreover, while the various embodiments described above are discussed in the context of transcutaneous placement of an analyte sensor, the scope of the present invention may also include implantable sensors.

Indeed, there is provided an apparatus including an inserter in one embodiment of the present invention including a housing for placement on a patient, the housing comprising a cavity, an introducer positioned at least substantially in the cavity, the introducer configured for displacement substantially within the cavity of the housing, and a sensor coupled to the introducer and at least substantially positioned in the cavity for transcutaneous deployment in the patient.

The sensor may be an electrochemical sensor.

In one embodiment, a transmitter unit may be provided mountable to the housing, where the transmitter unit is configured to be in electrical contact with the sensor. Further, the transmitter unit may be configured to receive a signal corresponding to an analyte level detected by the sensor, and to transmit the signal. Additionally, in a further embodiment, the transmitter unit may includes a wireless communication unit for wireless transmission of the signal, where the wireless communication unit may include one or more of an radio frequency (RF) communication unit, a Bluetooth communication unit, an infrared communication unit, an 801.11x communication unit, or a Zigbee communication unit.

In a further embodiment, the introducer may be configured with a spring mechanism such that the displacement force of the introducer substantially corresponds to the spring mechanism. In such embodiment, the spring mechanism may include a torsion coil coupled to the introducer within the housing. Alternatively, the introducer may be configured to be substantially flexible and is provided with the spring mechanism.

Also, a plunger may be provided and coupled to the introducer to trigger the spring mechanism of the introducer.

In yet a further embodiment, a portion of the sensor may be fixedly positioned within the cavity upon deployment of the sensor by the introducer.

Also, the introducer may be substantially completely positioned within the cavity of the housing after the deployment of the sensor.

In one embodiment, the sensor includes a glucose sensor.

A method of introducing a sensor in accordance with another embodiment of the present invention includes the steps of placing a housing on a skin of a patient, the housing including a sensor and a sensor introducer, the housing further configured to removably couple to a transmitter, activating an insertion mechanism coupled to the housing to insert the sensor through the skin of the patient, so that the transmitter may be in electrical contact with the sensor.

The method in a further embodiment may include the step of detecting an analyte level of the patient by the sensor, and providing the detected analyte level to the transmitter for signal transmission, where the signal transmission includes wireless transmission of a signal corresponding to the analyte level.

An insertion kit in ye another embodiment of the present invention includes a housing for transcutaneously inserting an analyte sensor and monitoring an analyte level of a patient, the housing having a cavity and the analyte sensor therein, the housing for placement on the patient, an introducer positioned at least substantially in the cavity and coupled to the analyte sensor, the introducer configured to insert the analyte sensor through a skin of the patient to a predetermined depth, where the inserted analyte sensor is in fluid contact with the analyte of the patient.

Various other modifications and alterations in the structure and method of operation of this invention will be apparent to those skilled in the art without departing from the scope and spirit of the invention. Although the invention has been described in connection with specific preferred embodiments, it should be understood that the invention as claimed should not be unduly limited to such specific embodiments. It is intended that the following claims define the scope of the present invention and that structures and methods within the scope of these claims and their equivalents be covered thereby.

1. An apparatus including an inserter, comprising:
a housing for placement on a patient, the housing comprising a cavity;
an introducer positioned at least substantially in the cavity, the introducer configured for displacement substantially within the cavity of the housing; and
a sensor coupled to the introducer and at least partially positioned in the cavity for transcutaneous deployment in the patient;
wherein at least a portion of the sensor and the introducer are retained within the cavity of the housing after deployment of the sensor in the patient.

2. The apparatus of claim 1 wherein the sensor is an electrochemical sensor.

3. The apparatus of claim 1 further including a transmitter unit mountable to the housing, and further, wherein the transmitter unit is configured to be in electrical contact with the sensor.

4. The apparatus of claim 3 wherein the transmitter unit is configured to receive a signal corresponding to an analyte level detected by the sensor, and further, to transmit the signal.

5. The apparatus of claim 4 wherein the transmitter unit includes a wireless communication unit for wireless transmission of the signal.

6. The apparatus of claim 5 wherein the wireless communication unit includes one or more of an rf communication unit, a Bluetooth communication unit, an infrared communication unit, an 801.11x communication unit, or a Zigbee communication unit.

7. The apparatus of claim 1 wherein the introducer is configured with a spring mechanism such that the displacement force of the introducer substantially corresponds to the spring mechanism.

8. The apparatus of claim 7 wherein the spring mechanism includes a torsion coil coupled to the introducer within the housing.

9. The apparatus of claim 7 wherein the introducer is substantially flexible and is provided with the spring mechanism.

10. The apparatus of claim 9 further including a plunger coupled to the introducer to trigger the spring mechanism of the introducer.

11. The apparatus of claim 1 wherein a portion of the sensor is fixedly positioned within the cavity upon deployment of the sensor by the introducer.

12. The apparatus of claim 1 wherein the introducer is substantially completely positioned within the cavity of the housing after the deployment of the sensor.

13. The apparatus of claim 1 wherein the sensor is a glucose sensor.

14. A method of introducing a sensor, comprising:
placing a housing on a skin of a patient, the housing including a sensor and a sensor introducer, the housing further configured to removably couple to a transmitter;
activating an insertion mechanism coupled to the housing to insert the sensor through the skin of the patient; and
retaining at least a portion of the sensor and the insertion mechanism within the housing after activating the insertion mechanism.

15. The method of claim 14 wherein the transmitter is in electrical contact with the sensor.

16. The method of claim 15 further including the step of detecting an analyte level of the patient by the sensor, and providing the detected analyte level to the transmitter for signal transmission.

17. The method of claim 16 wherein the signal transmission includes wireless transmission of a signal corresponding to the analyte level.

18. The method of claim 14 wherein the sensor is a glucose sensor.

19. An insertion kit, comprising:
a housing for transcutaneously inserting an analyte sensor and monitoring an analyte level of a patient, the housing having a cavity and the analyte sensor therein, the housing for placement on the patient; and
an introducer positioned at least substantially in the cavity and coupled to the analyte sensor, the introducer configured to insert the analyte sensor through a skin of the patient to a predetermined depth;
wherein at least a portion of the analyte sensor and the introducer are retained within the cavity of the housing after deployment of the sensor in the patient.

20. The kit of claim 19 wherein the inserted analyte sensor is in fluid contact with the analyte of the patient.
